(19) United States
(12) Patent Application Publication
(10) Pub. No.: US 2024/0068915 A1
(43) Pub. Date: Feb. 29, 2024

(54) ANALYSIS APPARATUS, ANALYSIS SYSTEM, ANALYSIS METHOD, CONTROL PROGRAM, AND RECORDING MEDIUM

(71) Applicant: KYOCERA Corporation, Kyoto-shi, Kyoto (JP)

(72) Inventors: Hanako ISHIDA, Kyoto (JP); Shinichi ABE, Kyoto (JP); Daisuke UEYAMA, Kyoto-shi, Kyoto (JP); Etsuro SHIMIZU, Kyoto-shi, Kyoto (JP)

(21) Appl. No.: 18/284,385

(22) PCT Filed: Mar. 15, 2022
(86) PCT No.: PCT/JP2022/011672

§ 371 Date: Sep. 27, 2023

(30) Foreign Application Priority Data
Mar. 31, 2021 (JP) 2021-060977

(51) Int. Cl. G01N 1/24; G01N 33/52

(52) U.S. Cl. CPC G01N 1/24; G01N 33/528; G01N 2001/245

(57) ABSTRACT

To improve accuracy of gas analysis. An analysis apparatus is placed on a toilet bowl and collects and analyzes a sample gas in the toilet bowl. The analysis apparatus includes a gas flow generator including a sucker and a discharger and generating a gas flow of the sample gas passing through the inside of the toilet bowl, and an analyzer that analyzes a component contained in the sample gas.

### TECHNICAL FIELD

The present disclosure relates to an analysis apparatus, an analysis system, an analysis method, and the like for analyzing gas discharged from a subject's body.

### BACKGROUND OF INVENTION

As described in Patent Document 1, a system for detecting odorous gas emitted from feces discharged by an examinee is known.

### CITATION LIST

### Patent Literature

Patent Document 1: JP 2016-145809 A

### SUMMARY

According to an aspect of the present disclosure, an analysis apparatus includes a gas flow generator including a sucker configured to suck a sample gas in a toilet bowl and a discharger configured to discharge the sample gas sucked by the sucker toward the inside of the toilet bowl, and configured to generate a gas flow of a sample gas passing through the inside of the toilet bowl, and an analyzer configured to analyze a component contained in the sample gas.

According to an aspect of the present disclosure, an analysis method includes generating a gas flow passing through the inside of a toilet bowl by sucking a sample gas in the toilet bowl and discharging the sample gas sucked toward the inside of the toilet bowl, collecting a sample gas from the gas flow, and analyzing a component contained in the sample gas collected.

According to each aspect of the present disclosure, an analysis apparatus may be implemented by a computer. In this case, a control program of the analysis apparatus and the analysis system configured to cause a computer to implement the analysis apparatus by causing the computer to operate as each unit (software element) included in the analysis apparatus, and a computer-readable recording medium recording the control program are also included within the scope of the present disclosure.

### BRIEF DESCRIPTION OF THE DRAWINGS

FIG. 1 is an external view illustrating a configuration of an analysis system according to an embodiment of the present disclosure.

FIG. 2 includes external views illustrating part of the configuration illustrated in FIG. 1 viewed from other viewpoints.

FIG. 3 is a block diagram illustrating a configuration of an analysis apparatus illustrated in FIG. 1.

FIG. 4 is a schematic diagram illustrating an example of a configuration of a gas flow generator.

FIG. 5 includes schematic diagrams illustrating other examples of configurations of the gas flow generator.

FIG. 6 is a schematic diagram of a gas collector and an analyzer included in the analysis apparatus illustrated in FIG. 1.

FIG. 7 includes diagrams illustrating a flow of gas around a suction tube.

FIG. 8 is a flowchart illustrating an example of a flow of processing executed in the analysis system.

FIG. 9 is an external view illustrating a configuration of another example of a blocker.

FIG. 10 is an external view illustrating a configuration of still another example of the blocker.

FIG. 11 is a schematic diagram illustrating another example of the gas flow generator.

FIG. 12 is a cross-sectional view illustrating still another example of a gas flow generator.

FIG. 13 is a cross-sectional view illustrating another example of the gas flow generator.

FIG. 14 is a schematic diagram illustrating a configuration of a gas collector and an analyzer according to another embodiment.

FIG. 15 is a schematic diagram illustrating a configuration of a gas flow generator, a gas collector, and an analyzer according to another embodiment.

FIG. 16 is a schematic diagram illustrating a configuration of a gas flow generator, a gas collector, and an analyzer according to another embodiment.

FIG. 17 is a schematic diagram illustrating another example of a gas flow generator 12.

FIG. 18 includes a graph and a table showing results of experiments according to Example 3.

FIG. 19 includes a graph and a table showing results of experiments according to Example 1.

FIG. 20 includes a graph and a table showing results of experiments according to Example 2.

FIG. 21 includes a graph and a table showing results of experiments according to Example 3.

FIG. 22 is a diagram illustrating analysis results according to Example 4.

FIG. 23 is a schematic diagram illustrating an example of arrangement of a gas flow generator in experiments according to Example 5.

FIG. 24 is a table showing results of the experiments according to Example 5.

### DESCRIPTION OF EMBODIMENTS

### First Embodiment

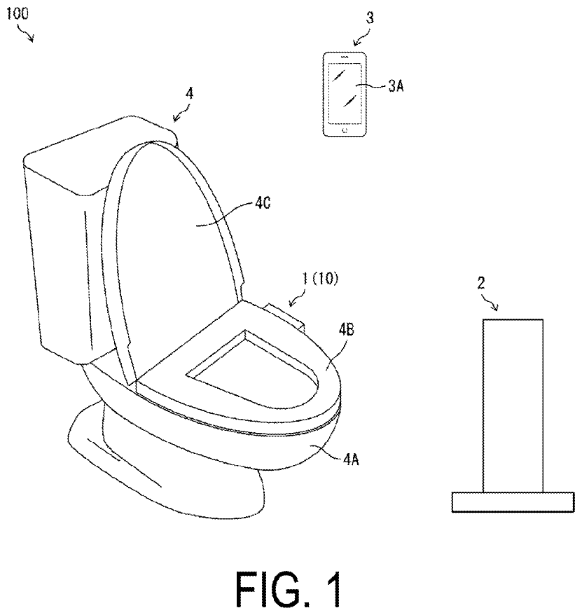

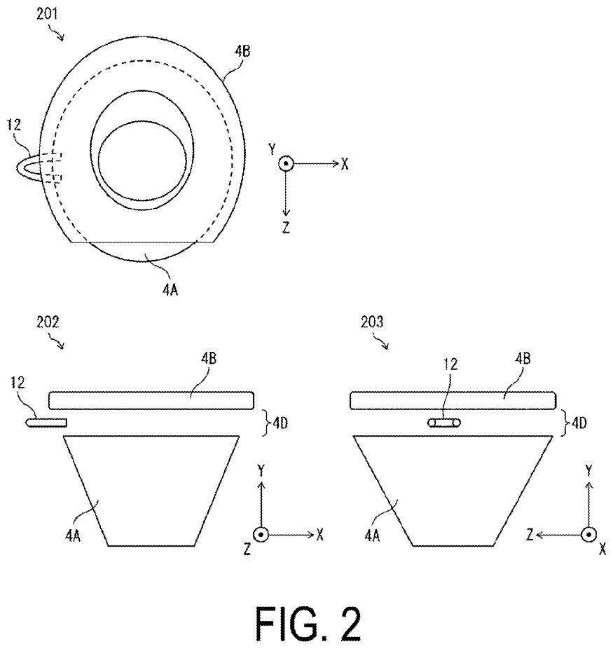

An embodiment of the present disclosure will be described in detail below. FIG. 1 is an external view illustrating a configuration of an analysis system 100 according to the embodiment of the present disclosure. FIG. 2 includes external views illustrating part of the configuration illustrated in FIG. 1 viewed from other viewpoints. However, for convenience of explanation, drawings referred to this specification are schematic diagrams illustrating only some members in a simplified manner for describing the embodiments. Thus, the analysis system 100 may include any constituent members not illustrated in the drawings to which this specification refers. The dimensions of the members in the drawings do not faithfully represent the actual dimensions of the constituent members, the dimension ratios of the members, or the like.

The analysis system 100 as illustrated in FIG. 1 may be referred to as a “gas detection system” or a “gas analysis system”. As illustrated in FIG. 1, the analysis system 100 includes an analysis apparatus 1, a server apparatus 2, and an electronic device (terminal device) 3. As illustrated in FIG. 1, the analysis apparatus 1 is placed on a toilet 4. The toilet 4 may be, but is not limited to, a flush toilet. The toilet 4 includes a toilet bowl 4A, a toilet seat 4B, and a lid 4C. The analysis apparatus 1 may be placed at any position on the toilet 4. As an example, the analysis apparatus 1 may be placed near a side portion of the toilet bowl 4A or the toilet seat 4B of the toilet 4. Part of the analysis apparatus 1 may be embedded in the toilet bowl 4A or the toilet seat 4B. Feces of an examinee (subject) may be discharged into the toilet bowl 4A of the toilet 4. The analysis apparatus 1 can acquire gas emitted from feces discharged into the toilet bowl 4A as a sample gas. The analysis apparatus 1 can detect a type, concentration, and the like of gas contained in the sample gas. The analysis apparatus 1 can transmit a detection result and the like to the server apparatus 2.

The toilet 4 may be installed in a toilet room of a house, a hospital, or the like. The toilet 4 may be used by the examinee. As described above, the toilet 4 includes the toilet bowl 4A and the toilet seat 4B. Feces of the examinee may be discharged into the toilet bowl 4A.

A view in reference numeral 201 in FIG. 2 illustrates the toilet 4 viewed from the top. A view in reference numeral 201 in FIG. 2 illustrates the toilet 4 viewed from a +Z-axis side of the view in reference numeral 201. A view in reference numeral 201 in FIG. 2 illustrates the toilet 4 viewed from the top. In the views in FIG. 2, a configuration of the toilet 4 is partially omitted and exaggerated. For example, in FIG. 2, the toilet bowl 4A and the toilet seat 4B are illustrated as not being connected, but actually the toilet bowl 4A and the toilet seat 4B are at least partially connected. The toilet bowl 4A includes an upper edge 4A1, as illustrated in FIG. 2. The upper edge 4A1 may have an oval ring shape when viewed from the top. The toilet seat 4B may include a U-shaped portion when viewed from the top. The toilet seat 4B may include, for example, four cushions on a surface facing the upper edge 4A1. When the toilet seat 4B is placed on the toilet bowl 4A, contact between the cushions and the upper edge 4A1 may create a space 4D between the upper edge 4A1 of the toilet bowl 4A and the toilet seat 4B.

The server apparatus 2 is an apparatus communicably connected to the analysis apparatus 1 and the electronic device 3, and can receive information indicating an analysis result obtained by the analysis apparatus 1 from the analysis apparatus 1 by wireless communication or wired communication. The server apparatus 2 can estimate the health condition of the examinee based on the analysis result obtained by the analysis apparatus 1 and transmit (output) information indicating the estimated health condition to the electronic device 3. A method of the estimation may be, for example, a method using a learned AI or the like capable of estimating the health condition of the examinee based on the type, concentration, and the like of the gas contained in the sample gas.

The electronic device 3 illustrated in FIG. 1 is, for example, a smartphone used by the examinee. However, the electronic device 3 is not limited to a smartphone and may be any electronic device. When the electronic device 3 is brought into the toilet room with the examinee, the electronic device 3 may be present inside the toilet room as illustrated in FIG. 1. However, for example, when the examinee does not bring the electronic device 3 into the toilet room, the electronic device 3 may be present outside the toilet room. The electronic device 3 can receive the information indicating the health condition of the examinee from the server apparatus 2 by wireless communication or wired communication. The electronic device 3 can present the health information of the examinee to the examinee by displaying the received information on a display 3A. The display 3A may include a display capable of displaying characters and the like and a touch screen capable of detecting contact of a user's (examinee's) finger or the like. This display may include a display device such as a liquid crystal display (LCD), an organic electro-luminescence display (OELD), or an inorganic electro-luminescence display (IELD). A detection method of this touch screen may be any method such as a capacitive method, a resistive film method, a surface acoustic wave method (or an ultrasonic method), an infrared method, an electromagnetic inductive method, or a load detection method.

Analysis Apparatus 1

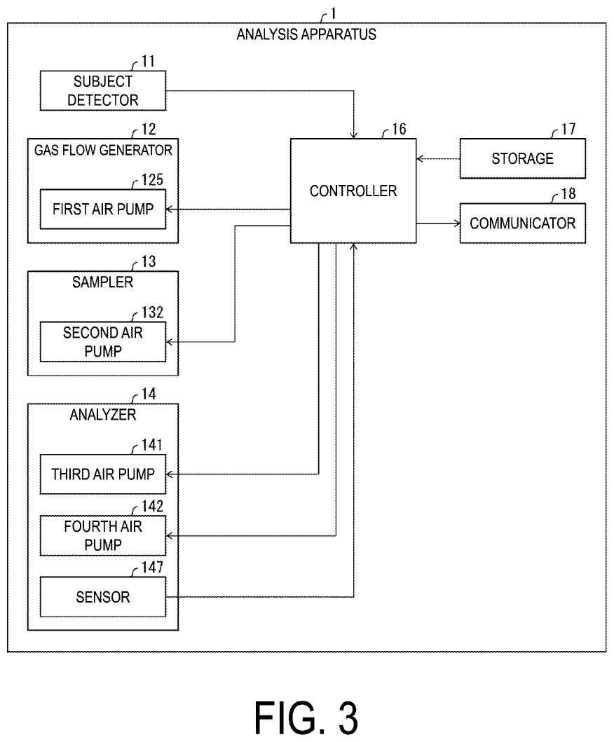

FIG. 3 is a block diagram illustrating a configuration of the analysis apparatus 1 illustrated in FIG. 1. As described above, the analysis apparatus 1 is placed on the toilet bowl 4A, detects the type, concentration, and the like of the gas contained in the sample gas acquired from the feces of the examinee, and transmits the information indicating the detection result to the server apparatus 2. In other words, the analysis apparatus 1 can analyze the collected sample gas. As illustrated in FIGS. 1 to 3, the analysis apparatus 1 includes a subject detector 11, a gas flow generator 12, a gas collector (sampler) 13, an analyzer 14, a blocker 15, a controller 16, a storage 17, and a communicator 18.

Subject Detector 11

The subject detector 11 may include at least one of an image camera, an individual identification switch, an infrared sensing device, a pressure sensing device, and the like. The subject detector 11 outputs a detection result to the controller 16. Besides these, the subject detector 11 may include any sensing device for authenticating the examinee. Examples of such sensing devices include a load sensing device that detects body weight, a sensing device that detects sitting height, a sensing device that detects pulse, a sensing device that detects blood flow, a sensing device that detects face, and a sensing device that detects voice.

For example, when the subject detector 11 includes an infrared sensing device, the infrared sensing device can detect that the examinee has entered the toilet room by emitting infrared light and detecting the light reflected from an object. The subject detector 11 outputs a signal indicating that the examinee has entered the toilet room to the controller 16 as a detection result.

For example, when the subject detector 11 includes a pressure sensing device, the subject detector 11 can detect that the examinee has sat on the toilet seat 4B by detecting pressure applied to the toilet seat 4B as illustrated in FIG. 1. The subject detector 11 outputs a signal indicating that the examinee has sat on the toilet seat 4B to the controller 16 as a detection result.

For example, when the subject detector 11 includes a pressure sensing device, the subject detector 11 can detect that the examinee has stood up from the toilet seat 4B by detecting a decrease in pressure applied to the toilet seat 4B as illustrated in FIG. 1. The subject detector 11 outputs a signal indicating that the examinee has stood up from the toilet seat 4B to the controller 16 as a detection result.

The subject detector 11 may detect that the examinee has defecated. The subject detector 11 outputs a signal indicating that the examinee has defected to the controller 16 as a detection result.

For example, when the subject detector 11 includes an image camera, an individual identification switch, and the like, the subject detector 11 collects data such as a face image, sitting height, and weight. The subject detector 11 specifies/identifies from the collected data and detects an individual. The subject detector 11 outputs a signal indicating the specified/identified individual to the controller 16 as a detection result.

For example, when the subject detector 11 includes an individual identification switch and the like, the subject detector 11 specifies (detects) an individual based on operation of the individual identification switch. In this case, personal information may be registered (stored) in advance in the controller 16. The subject detector 11 outputs a signal indicating the specified individual to the controller 16 as a detection result.

Gas Flow Generator 12

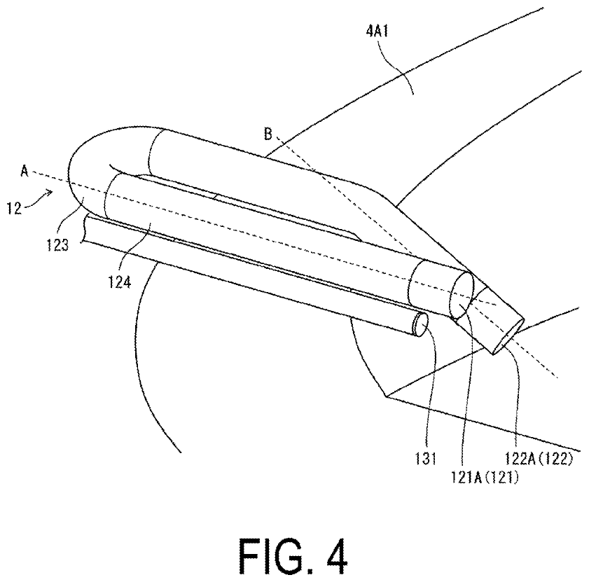

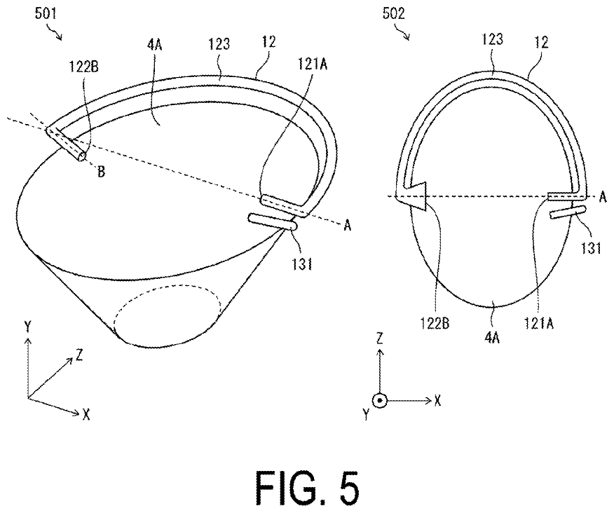

FIG. 4 is a schematic diagram illustrating an example of a configuration of the gas flow generator 12. FIG. 5 includes schematic diagrams illustrating other examples of configurations of the gas flow generator 12. The gas flow generator 12 generates a circulating flow of gas containing a sample gas (a gas flow of the sample gas) in the toilet 4. As illustrated in FIGS. 2 and 4, the gas flow generator 12 may be provided in the space 4D between the toilet bowl 4A and the toilet seat 4B. However, a position where the gas flow generator 12 is provided is not limited to the position described above. For example, at least part of the gas flow generator 12 may be embedded in the toilet 4. In other words, the gas flow generator 12 may be provided so as to be integrated with the toilet 4. As illustrated in FIGS. 2 to 4, the gas flow generator 12 may include a sucker 121, a discharger 122, a pipe 123, and a gas flow generating device 124.

The sucker 121 sucks a sample gas in the toilet bowl 4A. As illustrated in FIG. 4, the gas flow generator 12 includes a first end 121A as an example of the sucker 121. The discharger 122 discharges the sample gas sucked by the sucker 121 into the toilet bowl 4A. As illustrated in FIG. 4, the gas flow generator 12 includes a second end 122A as an example of the discharger 122. The first end 121A and the second end 122A are connected by the pipe 123. When the toilet seat 4B corresponding to the toilet bowl 4A is placed on the upper edge 4A1 of the toilet bowl 4A, the sucker 121 and the discharger 122 may be located in the space 4D between the toilet seat 4B and the upper edge 4A1 of the toilet bowl 4A.

In the following description, as an example, the gas flow generator 12 includes a first air pump 125 as an example of the gas flow generating device 124. However, the configuration of the gas flow generator 12 is not limited to that described above.

The first end 121A is one end portion of the gas flow generator 12, and is an end portion that sucks gas in the toilet bowl 4A. As illustrated in FIG. 4, the first end 121A has an opening portion that opens towards the interior of the toilet bowl 4A. In the following, a central axis parallel to a direction in which the opening portion of the first end 121A opens is referred to as “central axis A”. When the gas flow generator 12 is located in the space 4D between the toilet bowl 4A and the toilet seat 4B, the central axis A may be substantially parallel to a seat surface of the toilet seat 4B.

The second end 122A is the other end portion of the gas flow generator 12, and is an end portion that discharges the gas sucked from the first end 121A. As illustrated in FIG. 4, the second end 122A has an opening portion that opens towards the interior of the toilet bowl 4A. In the following, a central axis parallel to a direction in which the opening portion of the second end 122A opens is referred to as “central axis B”. When the gas flow generator 12 is placed on a back surface of the toilet seat 4B, the central axis B may be inclined from the direction parallel to the seat surface of the toilet seat 4B to a bottom of the toilet bowl 4A (−Z-axis direction).

As an example, as illustrated in FIG. 4, the first end 121A may be provided near a suction tube 131. The second end 122A may be provided near the first end 121A. As another example, the second end 122A may be provided at a position away from the first end 121A. For example, as indicated in reference numerals 501 and 502 in FIG. 5, a second end 122A may be provided at a position facing the first end 121A when the toilet bowl 4A is viewed from the top. However, the positions where the first end 121A and the second end 122A are located are not limited to the positions described above, and may be located at any positions.

As indicated in reference numeral 502 in FIG. 5, the first end 121A may be provided at a position spaced apart from a distal end of the suction tube 131. The central axis A parallel to the direction in which the opening portion of the first end 121A opens may form an angle with the suction tube 131. This reduces a possibility that a sample gas to be sucked by the suction tube 131 is sucked by the gas flow generator 12 from the first end 121A. As illustrated in reference numeral 502 in FIG. 5, a width of the second end 122B (a diameter of the opening) may be wider than a width of the first end 121A. Thus, a gas flow is discharged from the second end 122B to a wider area. Therefore, even when a position of the examinee's feces or a position of the analysis apparatus 1 is not appropriate, a sample gas can be sucked from the suction tube 131.

The first end 121A may open towards the bottom of the toilet bowl 4A. At this time, the second end 122A may open in a direction parallel to the toilet seat 4B or may open in a direction toward the bottom of the toilet bowl 4A. Both the first end 121A and the second end 122A may be inclined towards the bottom of the toilet bowl 4A. An angle formed by the direction in which the first end 121A opens and the direction parallel to the toilet seat 4B forms an acute angle in FIG. 4, but this angle may be larger, for example, a right angle. That is, the first end 121A may open directly downward (−Y-axis direction illustrated in FIG. 2, etc.).

The pipe 123 is a hollow member that connects the first end 121A and the second end 121A. The shape of the pipe 123 may be, for example, cylindrical as illustrated in FIG. 4, but is not limited thereto. As illustrated in FIG. 4, the pipe 123 may be provided outside the toilet bowl 4A. The pipe 123 may be made of any material. For example, the pipe 123 may be made of a material such as metal or resin.

The first air pump 125 is a pump connected to the pipe 123, and sucks gas in the toilet bowl 4A from the first end 121A and discharges from the second end 121A via the inside of the pipe 123. The first air pump 125 may be a piezo pump, a motor pump, or the like.

A gas flow is generated in the toilet bowl 4A by sucking from the first end 121A and discharging from the second end 122A. A sample gas is emitted from the examinee's feces and then accumulates at the bottom of the toilet bowl 4A. However, the sample gas is swirled up by the gas flow generated by the gas flow generator 12 and flows upward of the toilet bowl 4A (toward the lid 4C), in particular, in a direction in which the suction tube 131 of the gas collector 13 described later is provided. Thus, the sample gas can be more efficiently collected from the suction tube 131.

The gas flow generator 12 sucks gas in the toilet bowl 4A and discharges the gas into the toilet bowl 4A. Therefore, a possibility that air outside the toilet bowl 4A flows into the toilet bowl 4A and a possibility that gas inside the toilet bowl 4A flows out of the toilet bowl 4A are reduced. Thus, the generation of the gas flow by the gas flow generator 12 reduces a possibility that a concentration of the sample gas in the toilet bowl 4A decreases.

By directing the central axis B of the second end 122A toward the bottom of the toilet bowl 4A, the gas flow generated by the gas flow generator 12 can swirl up the sample gas accumulated at the bottom of the toilet bowl 4A. Thus, the concentration of the sample gas around the suction tube 131 can be increased, so that the concentration of the gas contained in the sample gas can be measured with higher accuracy.

Gas Collector 13

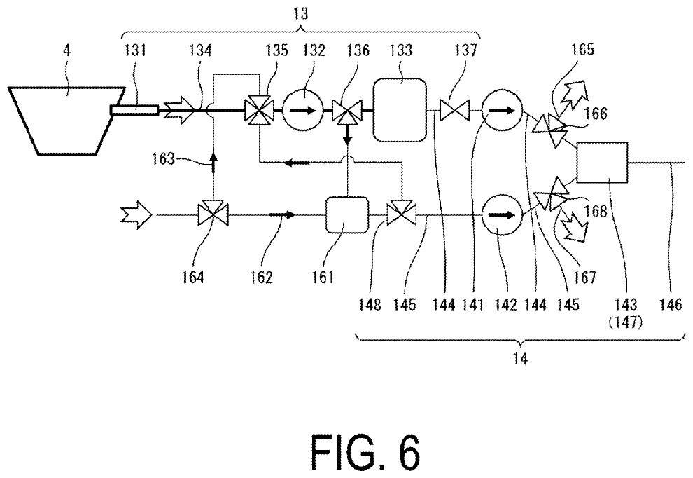

FIG. 6 is a schematic diagram of the gas collector 13 and the analyzer 14 included in the analysis apparatus 1 illustrated in FIG. 1. As illustrated in FIG. 6, the gas collector 13 includes the suction tube 131 having an opening portion and a flow path, a second air pump 132, and a reservoir tank (reservoir) 133. In the following description, the reservoir tank 133 is made, as an example, of a flexible material that can expand, contract, or deform to change an internal volume of the reservoir tank 133, depending on an amount of gas reserved in the reservoir tank 133.

The gas collector 13 collects gas emitted from feces discharged into the toilet bowl 4A as a sample gas. The gas collector 13 collects at least some of a gas flow generated by the gas flow generator as a sample gas. For example, the controller 16 described later generates a gas flow from the gas collector 13 to the reservoir tank 133 as illustrated in FIG. 6, whereby the gas collector 13 collects the gas emitted from the feces discharged into the toilet bowl 4A as a sample gas. As illustrated in FIG. 4, the gas collector 13 may be provided between the toilet seat 4B and the upper edge 4A1 of the toilet bowl 4A. For example, the gas collector 13 may be located on the back surface of the toilet seat 4B. In this case, part of the gas collector 13 may be located in the space 4D formed between the upper edge 4A1 of the toilet bowl 4A and the toilet seat 4B when the upper edge 4A1 of the toilet bowl 4A and the cushions 4B1 of the toilet seat 4B come into contact. For example, when the space 4D between the upper edge 4A1 and the toilet seat 4B is small, part of the gas collector 13 may be embedded in the toilet bowl 4A or the toilet seat 4B.

The suction tube 131 is a tube for supplying a sample gas from the toilet bowl 4A to the reservoir tank 133. As illustrated in FIG. 4, the suction tube 131 may be located between the toilet seat 4B and the upper edge 4A1, at a gently arc-shaped portion of the oval ring-shaped upper edge 4A1 on a right side when viewed from the top. However, the position of the suction tube 131 between the toilet seat 4B and the upper edge 4A1 is not limited thereto. The suction tube 131 may be located at any position of the oval ring-shaped upper edge 4A1 between the toilet seat 4B and the upper edge 4A1. For example, the suction tube 131 may be located at a protruding portion of the oval ring-shaped upper edge 4A1. For example, the suction tube 131 may be located at a gently arc-shaped portion of the oval ring-shaped upper edge 4A1 on the left side when viewed from the top. When the position of the suction tube 131 and the position of the reservoir tank 133 are separated, the suction tube 131 may be connected to the reservoir tank 133 in a housing 10 via a pipe-shaped member such as a resin tube or a metal or glass pipe.

The suction tube 131 may be cylindrical or prismatic in shape. In the present embodiment, the suction tube 131 has a cylindrical shape. However, the suction tube 131 may be of any shape. When the housing 10 for the analysis apparatus 1 is placed on a side portion of the toilet 4 as illustrated in FIG. 1, at least part of the gas collector 13 may be placed in the space 4D between the toilet bowl 4A and the toilet seat 4B as illustrated in FIG. 2. In this case, a central axis of the cylindrical suction tube 131 may be substantially parallel to the back surface of the toilet seat 4B as illustrated in FIG. 4. The suction tube 131 may be made of any material. For example, the suction tube 131 may be made of a material such as metal or resin.

The suction tube 131 of the gas collector 13 has the opening portion for supplying a sample gas from the toilet bowl 4A into the suction tube 131. This opening portion may be located in the space 4D between the toilet seat 4B and the upper edge 4A1 of the toilet bowl 4A when the toilet seat 4B corresponding to the toilet bowl 4A is placed on the upper edge 4A1 of the toilet bowl 4A.

A flow path 134 of the suction tube 131 is connected to the reservoir tank 133 as illustrated in FIG. 6 as a predetermined tank. The flow path 134 is formed inside the suction tube 131. The flow path 134 supplies a sample gas that flows through the opening portion of the suction tube 131 into the reservoir tank 133 as illustrated in FIG. 6. The flow path 134 is formed such that a central axis of the flow path 134 is aligned with the central axis of the suction tube 131. However, at least part of the flow path 134 may be curved.

The opening portion of the suction tube 131 supplies the sample gas into the flow path. In the present embodiment, the opening portion of the suction tube 131 is located between the toilet seat 4B and the upper edge 4A1 of the toilet bowl 4A as illustrated in FIG. 4. The opening portion of the suction tube 131 faces the inside of the toilet bowl 4A. Part of the suction tube 131 may be embedded in the toilet bowl 4A or toilet seat 4B. In the present embodiment, the suction tube 131 does not protrude inside the toilet bowl 4A, as illustrated in FIG. 4. With such a configuration, adhesion of feces, urine, and the like to the suction tube 131 can be reduced.

As illustrated in FIG. 6, the second air pump 132 is provided between the suction tube 131 and the reservoir tank 133. The second air pump 132 supplies a sample gas in the toilet bowl 4A to the reservoir tank 133 via the suction tube 131 under control of the controller 16. The second air pump 132 illustrated in FIG. 6 may be configured by a piezo pump, a motor pump, or the like. The second air pump 132 may also be used when reserving a purge gas in a purge gas reservoir tank 161, as will be described later.

As illustrated in FIG. 6, the reservoir tank 133 is connected to the suction tube 131. A valve 135 may be provided between the suction tube 131 and the second air pump 132. A valve 136 may be provided between the second air pump 132 and the reservoir tank 133. The valve 135 may be, for example, an electromagnetically driven, a piezo-driven, or a motor-driven valve. The valve 135 may switch the state of connection between the reservoir tank 133 and the suction tube 131 to a state in which the reservoir tank 133 and the suction tube 131 are connected, or a state in which the reservoir tank 133 and the suction tube 131 are not connected, based on control by the controller 16 as illustrated in FIG. 3. As described above, the reservoir tank 133 may be made of a flexible material such as resin or resin coated with metal that can be deformed according to an amount of gas reserved therein. When the reservoir tank 133 is made of a flexible material, the sample gas remaining therein can be reduced, thereby reducing a possibility of contact between a newly collected sample gas and the previously collected sample gas. The reservoir tank 133 may be made of a material such as metal or resin that does not deform according to the amount of gas reserved therein.

A sample gas is supplied to the reservoir tank 133 through the suction tube 131. The reservoir tank 133 can store the sample gas. The sample gas stored in the reservoir tank 133 is supplied to a chamber 143 via a third air pump 141. A valve 137 may be provided between the reservoir tank 133 and the third air pump 141. The third air pump 141 can supply a predetermined volume of the sample gas to a sensor 147. The reservoir tank 133 may be configured by, for example, a tank having a rectangular parallelepiped shape, a cylindrical shape, a bag shape, or a shape that fills space between various components housed inside the housing 10. The reservoir tank 133 may be provided with a heater for heating a sample gas.

An adsorbent may be placed inside the reservoir tank 133. The adsorbent may contain several types of materials depending on application. The adsorbent may include, for example, at least one of activated carbon, silica gel, zeolite, and molecular sieve. The adsorbent may be of multiple types and may include a porous material. The adsorbent may adsorb gases contained in a sample gas that are not to be detected. Examples of adsorbents that adsorb gases that are not to be detected include silica gel and zeolite. The sample gas may be concentrated in the reservoir tank 133. In this case, the adsorbent may adsorb a gas to be detected contained in the sample gas. Examples of adsorbents that adsorb the gas to be detected include activated carbon and molecular sieve. However, the combination of these adsorbents may be appropriately changed depending on a polarity of gas molecules to be adsorbed.

Analyzer 14

The analyzer 14 analyzes components contained in the sample gas collected by the gas collector 13. As illustrated in FIG. 6, the analyzer 14 may include the third air pump 141, a fourth air pump 142, the chamber 143, a flow path 144, a flow path 145, a discharge path 146, and the sensor 147.

As illustrated in FIG. 6, the third air pump 141 may be provided in the flow path 144 connecting the reservoir tank 133 and the chamber 143. The third air pump 141 supplies the sample gas stored in the reservoir tank 133 to the chamber 143 under control of the controller 16. An arrow illustrated in the third air pump 141 indicates a direction in which the third air pump 141 feeds the sample gas. The third air pump 141 may be configured by a piezo pump, a motor pump, or the like. A gas flow generated by operating the gas flow generator 12 may be directly supplied to the chamber 143 by operating the third air pump 141.

As illustrated in FIG. 6, the fourth air pump 142 may be provided in the flow path 145 connecting the chamber 143 and an opening portion provided in the toilet room. The fourth air pump 142 supplies a purge gas to the chamber 143 for removing the sample gas after detection processing by the sensor 147 under control of the controller 16. An arrow illustrated in the fourth air pump 142 indicates a direction in which the fourth air pump 142 feeds the purge gas. The fourth air pump 142 may be configured by a piezo pump, a motor pump, or the like. A valve 148 may also be provided in the flow path 145.

As illustrated in FIG. 6, the analyzer 14 may include the discharge path 146 for discharging a discharge gas from the chamber 143 to the outside. This discharge gas may include the sample gas after the detection processing and the purge gas. The flow path 144, the flow path 145, and the discharge path 146 may be configured by tubular members such as resin tubes or metal or glass pipes.

As illustrated in FIG. 6, the chamber 143 has the sensor 147 therein. The chamber 143 may have multiple sensors 147. The chamber 143 may be divided into multiple chambers. The sensors 147 may be placed in the divided chambers 143, respectively. The multiple divided chambers 143 may be connected to each other. The flow path 144 is connected to the chamber 143. The sample gas is supplied to the chamber 143 from the flow path 144. The flow path 145 is connected to the chamber 143. The purge gas is supplied to the chamber 143 from the flow path 145. The discharge path 146 is connected to the chamber 143. The chamber 143 discharges the sample gas after the detection processing and the purge gas through the discharge path 146. The chamber 143 may be made of a material such as metal or resin.

As illustrated in FIG. 6, the sensor 147 is placed in the chamber 143. The sensor 147 outputs a detection signal indicating a voltage value corresponding to a concentration of a specific gas to the controller 16 as illustrated in FIG. 3. The sensor 147 may output a detection signal to the controller 16 each time a predetermined volume of the sample gas and a predetermined volume of the purge gas are alternately supplied to the sensor 147. The specific gas includes a specific gas to be detected and a specific gas other than the specific gas to be detected. When a sample gas is a gas emitted from feces, examples of the specific gas to be detected include methane, hydrogen, carbon dioxide, methyl mercaptan, hydrogen sulfide, acetic acid, and trimethylamine. When the sample gas is the gas emitted from the feces, examples of the specific gas other than the specific gas to be detected include ammonia and water. Each of the multiple sensors 147 can output a voltage corresponding to the concentration of at least one of these gases to the controller 16 as illustrated in FIG. 3. The sensor 147 may be a semiconductor sensing device, a catalytic combustion sensing device, an electrochemical sensing device, a solid electrolyte sensing device, or the like.

Other Configurations of Gas Collector 13 and Analyzer 14

The gas collector 13 and the analyzer 14 included in the analysis apparatus 1 may have other configurations. For example, as illustrated in FIG. 6, the analysis apparatus 1 may include the purge gas reservoir tank 161 for reserving a purge gas upstream of the fourth air pump 142. In this case, the flow path 145 connects the fourth air pump 142 and the purge gas reservoir tank 161. A flow path 162 that connects the purge gas reservoir tank 161 and the outside such as a toilet room may be connected upstream of the purge gas reservoir tank 161. As illustrated in FIG. 6, the purge gas reservoir tank 161 may be connected to the reservoir tank 133. In this case, a flow path 163 connected to the flow path 134 is provided downstream of the purge gas reservoir tank 161, and a valve 164 is provided between the flow path 134 and the flow path 163.

As illustrated in FIG. 6, the flow path 144 may be connected to a flow path 165 that is connected to the outside. A valve 166 is provided between the flow path 144 and the flow path 165. The flow path 145 may be connected to a flow path 167 that is connected to the outside. A valve 168 may be provided between the flow path 145 and the flow path 167.

By opening the valve 164, the valve 135, and the valve 136 and driving the second air pump 132, a purge gas is sucked from the outside through the flow path 163 and the flow path 134 and reserved in the purge gas reservoir tank 161. After the measurement of the sample gas by the sensor 147 is completed, the purge gas is supplied to the reservoir tank 133 by driving the second air pump 132 with the valve 148, the valve 135, and the valve 136 open. Thereafter, by driving the third air pump 141 with the valve 165 open, the purge gas supplied to the reservoir tank is discharged to the outside through the flow path 165 together with the sample gas remaining in the reservoir tank 133. Thus, the reservoir tank 133 is cleaned.

In the gas collector 13 and the analyzer 14, the reservoir tank 133 may be cleaned before collection of a sample gas. First, the previously collected past sample gas remaining in the reservoir tank 133 is discharged. Specifically, by closing the valve 136 upstream of the reservoir tank 133 and operating the third air pump 141, the sample gas remaining in the reservoir tank 133 is discharged from the flow path 165. Subsequently, by closing the valve 137 downstream of the reservoir tank 133 and operating the second air pump 132, the purge gas is supplied from the purge gas reservoir tank 161 into the reservoir tank 133. Subsequently, by closing the valve 136 upstream of the reservoir tank 133 and operating the third air pump 141, the purge gas in the reservoir tank 133 is discharged from the flow path 165. Supplying the purge gas into the reservoir tank 133 and discharging the purge gas from the reservoir tank 133 may be performed multiple times. Finally, by closing the valve 137 downstream of the reservoir tank 133 and operating the second air pump 132, a new sample gas is collected from the toilet bowl 4A and supplied into the reservoir tank 133.

In the gas collector 13 and the analyzer 14, the purge gas reservoir tank 161 may be cleaned before collecting a purge gas. First, the past purge gas remaining in the purge gas reservoir tank 161 is discharged. Specifically, by closing the valve 164 upstream of the purge gas reservoir tank 161 and operating the fourth air pump 142, the purge gas in the purge gas reservoir tank 161 is discharged from the flow path 167. Subsequently, by closing the valve 148 downstream of the purge gas reservoir tank 161 and operating the second air pump 132, a purge gas is supplied into the purge gas reservoir tank 161. Subsequently, by closing the valve 164 upstream of the purge gas reservoir tank 161 and operating the fourth air pump 142, the purge gas in the purge gas reservoir tank 161 is discharged from the flow path 167. Supplying a purge gas into the purge gas reservoir tank 161 and discharging the purge gas from the purge gas reservoir tank 161 may be performed multiple times. Finally, by closing the valve 148 downstream of the purge gas reservoir tank 161 and operating the second air pump 132, a purge gas is supplied into the purge gas reservoir tank 161. A purge gas may be reserved in the purge gas reservoir tank 161 at a time when air in a space where the purge gas is collected is clean, for example, other than when the examinee defecates and when a sample gas is reserved.

Blocker 15

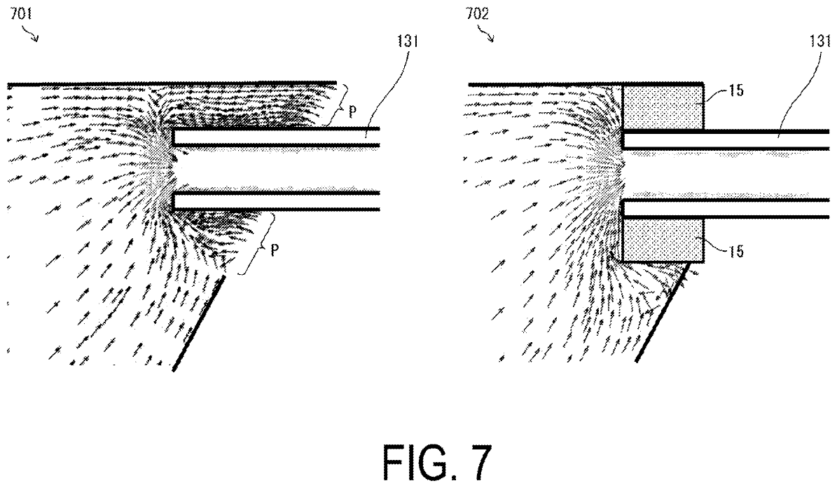

FIG. 7 includes diagrams, each illustrating a flow of gas around the suction tube 131. Reference numeral 701 in FIG. 7 indicates the flow of gas in the toilet 4 without the blocker 15, and reference numeral 702 indicates the flow of gas in the toilet 4 with the blocker 15. The blocker 15 may be provided on the toilet seat 4B to close or narrow a gap P between the toilet seat 4B and the toilet bowl 4A. As indicated in reference numeral 702 in FIG. 7, the blocker 15 may block at least some of a gas flow generated by the gas flow generator 12.

The blocker 15 may be provided at least around the suction tube 131 of the gas collector 13. As an example, as indicated in reference numeral 702 in FIG. 7, the blocker 15 may be provided near the suction tube 131 and cover the suction tube 131. The blocker 15 may be made of, for example, rubber, and may close the gap P by deforming due to weight of the toilet seat 4B.

As indicated in reference numeral 701 in FIG. 7, when a sample gas is sucked from the suction tube 131 without the blocker 15 around the suction tube 131, some of the gas flows out through the gap P to the outside of the toilet bowl 4A. Here, as indicated in reference numeral 702 in FIG. 7, by providing the blocker 15 around the suction tube 131, movement of the sample gas inside the toilet bowl 4A from the toilet bowl 4A to the outside is blocked by the blocker 15. This reduces outflow of the sample gas from the toilet bowl 4A by a certain amount, thereby slowing down a rate at which a concentration of the sample gas in the toilet bowl 4A decreases.

Controller 16

The controller 16 controls operation of the components in the analysis apparatus 1. The controller 16 may analyze components contained in the sample gas collected by the gas collector 13.

The controller 16 may generate a gas flow in the toilet bowl 4A by controlling the first air pump 125. For example, the controller 16 may control the first air pump 125 to suck air containing a sample gas in the toilet bowl 4A from the first end 121A of the gas flow generator 12 and discharge it from the second end 122A of the gas flow generator 12 via the pipe 123. The controller 16 may control the first air pump 125 based on a detection result of the subject detector 11 after a predetermined time has elapsed since the examinee defecates.

The controller 16 controls the second air pump 132 to cause the gas collector 13 to suck a sample gas or a purge gas. For example, the controller 16 controls the second air pump 132 with the valve 137 closed to reserve the sample gas in the reservoir tank 133. Specifically, the controller 16 controls the second air pump 132 with the valve 137 closed to generate a gas flow from the gas collector 13 to the reservoir tank 133. Thus, the sample gas is sucked by the gas collector 13 and reserved in the reservoir tank 133. The controller 16 may clean the reservoir tank 133 by controlling the second air pump 132 and the third air pump 141 before sample gas collection or after sample gas analysis. Specifically, the controller 16 closes the valve 137 and operates the second air pump 132. Thus, the purge gas is supplied from the purge gas reservoir tank 161 to the reservoir tank 133. Thereafter, by operating the third air pump 141, the sample gas remaining in the reservoir tank 133 together with the purge gas is discharged from the reservoir tank 133 to the outside via the flow path 165. Thus, the reservoir tank 133 is cleaned.

The controller 16 causes the gas collector 13 to suck a sample gas, thereby storing the sample gas in the reservoir tank 133. The controller 16 may cause the gas collector 13 to suck a sample gas based on a detection result of the subject detector 11 after a predetermined time has elapsed since detecting that the examinee has sat on the toilet seat 4B. Alternatively, the controller 16 may cause the gas collector 13 to suck a sample gas after a predetermined time has elapsed since the first air pump 125 was activated. Thus, a gas flow is generated in the toilet bowl 4A, which swirls up the sample gas accumulated at the bottom, allowing the sample gas to be sucked with an increased concentration of the sample gas around the suction tube 131.

The controller 16 may continue to operate the first air pump 125 while operating the second air pump 132, or may deactivate the first air pump 125 before activating the second air pump 132. Deactivating the first air pump 125 before activating the second air pump 132 reduces a possibility that the gas flow generated by the gas flow generator 12 will interfere the suction of the sample gas from the suction tube 131.

According to a command from the controller 16, air in the toilet room outside the toilet bowl 4A, as illustrated in FIG. 1, is sucked as a purge gas and stored in the purge gas reservoir tank 161. For example, the controller 16 opens the valve 164, the valve 135, and the valve 136, and drives the second air pump 132. Accordingly, a purge gas is sucked from the outside through the flow path 163 and the flow path 134 and reserved in the purge gas reservoir tank 161.

The controller 16 controls the third air pump 141 and the fourth air pump 142 to alternately supply the sample gas stored in the reservoir tank 133 and the purge gas stored in the purge gas reservoir tank 161 to the chamber 143. The controller 16 acquires voltage values from the sensor 147 by alternately supplying the purge gas and the sample gas to the chamber 143. The controller 16 may acquire a detection signal indicating the voltage value each time a predetermined volume of the sample gas and a predetermined volume of the purge gas are alternately supplied to the sensor 147. The controller 16 detects (analyzes) a type and concentration of a gas contained in the sample gas based on voltage waveform data constituted by the detection signals in which the voltage values based on the acquired sample gas and purge gas are associated with time. For example, the controller 16 detects the type and concentration of the gas contained in the sample gas by machine learning of the voltage waveform. The controller 16 may transmit the type and concentration of the detected gas to the electronic device 3 via the communicator 18 as a detection result. As an example, the controller 16 may detect a concentration of CO₂ as the gas contained in the sample gas.

Storage 17

The storage 17 may be, for example, a semiconductor memory, a magnetic memory, or the like. The storage 17 stores various kinds of information, a program for operating the analysis apparatus 1, and the like. The storage 17 may function as a work memory.

The communicator 18 can communicate with the electronic device 3 as illustrated in FIG. 1. The communicator 18 may be capable of communicating with an external server. A communication method used in communication between the communicator 18 and the server apparatus 2 and the electronic device 3 may be a short-range wireless communication standard, a wireless communication standard for connection to a mobile phone network, or a wired communication standard. The short-range wireless communication standard may include, for example, WiFi (registered trade mark), Bluetooth (registered trade mark), infrared rays, and near field communication (NFC). The wireless communication standard for connection to the mobile phone network may include, for example, Long Term Evolution (LTE) or a fourth generation or higher mobile communication system. The communication method used in communication between the communicator 18 and the server apparatus 2 and the electronic device 3 may be a communication standard such as low power wide area (LPWA) or low power wide area network (LPWAN).

Example of Flow of Processing Executed in Analysis System 100

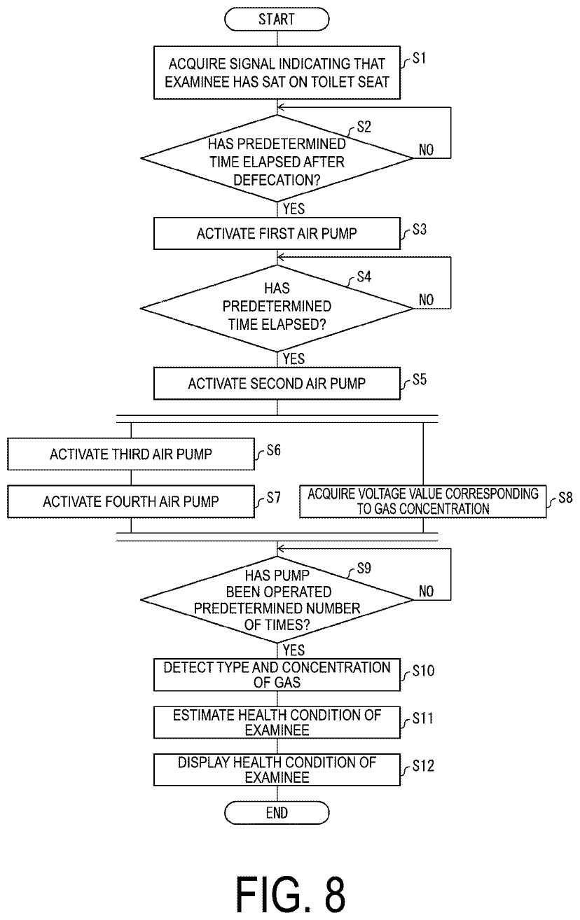

FIG. 8 is a flowchart illustrating an example of a flow of processing executed in the analysis system 100. The example of a flow of processing executed in the analysis system 100 will be described below with reference to FIG. 8. In the following description, the analysis apparatus 1 includes a pressure sensing device as the subject detector 11. A case in which the subject detector 11 can execute processing for detecting that the subject has sat on the toilet seat 4B and that the subject has defecated will be described as an example.

First, when the examinee sits on the toilet seat 4B in order to discharge feces into the toilet 4, the subject detector 11 detects that the examinee has sat on the toilet seat 4B. The controller 16 acquires a signal indicating that the examinee has sat on the toilet seat 4B from the subject detector 11 as a detection result (S1).

After S1, the subject detector 11 detects that the examinee has defecated. When a predetermined time elapses after the examinee defecates (YES in S2), the controller 16 activates the first air pump 125 of the gas flow generator 12 (S3: gas flow generation step). By operating the first air pump 125, a sample gas in the toilet bowl 4A is sucked into the gas flow generator 12 from the first end 121A and discharged from the second end 122A through the pipe 123. Thus, the sample gas accumulated at the bottom of the toilet bowl 4A is swirled up, and the concentration of the sample gas around the suction tube 131 increases.

After S3, when another predetermined time elapses (YES in S4), the controller 16 activates the second air pump 132 (S5: collection step). Thus, the sample gas in the toilet bowl 4A is sucked from the suction tube 131 and reserved in the reservoir tank 133. The time to activate the second air pump 132 may be, for example, 90 seconds after the examinee defecates. The controller 16 may control the second air pump 132 to suck the sample gas for 30 seconds at a gas supply rate of, for example, 1000 ml/min. The controller 16 may activate the second air pump 132 and the third air pump 141 to clean the reservoir tank 133 during a period from when the examinee defecates to when the second air pump 132 is activated.

After S5, the controller 16 activates the third air pump 141 (S6) to supply the sample gas in the reservoir tank 133 to the sensor 147 in the chamber 143. The gas supply rate when the third air pump 141 supplies the sample gas to the chamber 143 may be, for example, 50 ml/min. Subsequently, the controller 16 activates the fourth air pump 142 (S7) to supply a purge gas in the toilet room to the sensor 147 in the chamber 143. The controller 16 may operate the third air pump 141 and the fourth air pump 142 for two minutes each, for example. The controller 16 alternately executes processing of S6 and processing of S7 a predetermined number of times. The predetermined number of times may be, for example, three times.

While the processing of S6 and the processing of S7 are being executed, the controller 16 acquires, from the sensor 147, voltage values corresponding to the concentrations of the gas supplied to the sensor 147 (S8). That is, each time predetermined volumes of the sample gas and the purge gas are alternately supplied to the sensor 147, the controller 16 acquires a voltage value corresponding to the concentration of the gas contained in the sample gas or the purge gas.

When the processing of S6 and the processing of S7 have been executed the predetermined number of times (YES in S9), the controller 16 ends the operation of the third air pump 141 and the fourth air pump 142 and the acquisition of the voltage values from the sensor 147. The controller 16 creates voltage waveform data constituted by detection signals in which the acquired voltage values and the times at which the voltage values are acquired are associated with each other. The controller 16 detects a type and concentration of the gas contained in the sample gas based on the voltage waveform data (S10: analysis step). The controller 16 transmits data indicating the type and concentration of the gas contained in the detected sample gas to the server apparatus 2.

When the server apparatus 2 receives the data including the type and concentration of the gas contained in the sample gas of the examinee's feces received from the analysis apparatus 1, the server apparatus 2 estimates the health condition of the examinee based on the data (S11). The server apparatus 2 transmits information indicating the estimated health condition to the electronic device 3.

When receiving the information indicating the health condition of the examinee from the server apparatus 2, the electronic device 3 displays the received information on the display 3A (S12). Thus, the examinee can know his/her health condition from the electronic device 3.

Effects of Analysis Apparatus 1

As described above, the analysis apparatus 1 is placed on the toilet bowl 4A. The analysis apparatus 1 includes the gas flow generator 12 that includes the sucker 121 and the discharger 122 and generates a gas flow, the gas collector 13 that collects a sample gas contained in the gas flow, and the analyzer 14 that analyzes components contained in the sample gas.

According to the above configuration, the analysis apparatus 1 can generate a gas flow passing through the inside of the toilet bowl 4A using the sample gas in the toilet bowl 4A, collect the sample gas from the gas flow, and analyze the components contained in the sample gas. The gas flow can be generated using the sample gas itself to be analyzed. Thus, the analysis apparatus 1 can steadily collect the sample gas while avoiding dilution of the sample gas by air or the like outside the toilet bowl 4A. Therefore, analysis accuracy of the sample gas in the analysis apparatus 1 can be improved.

The gas flow generator 12 may include the pipe 123 including the first end 121A that is the sucker 121 and the second end 122A that is the discharger 122, and the gas flow generating device 124 located in the pipe 123. The gas flow generating device 124 may be the first air pump 125 capable of generating a gas flow from the first end 121A to the second end 122A. According to this configuration, the sample gas in the toilet bowl 4A is sucked from the first end 121A and discharged from the second end 122A. Thus, the sample gas in the toilet bowl 4A can be used to generate the gas flow in the toilet bowl 4A.

As described above, the gas collector 13 may include the second air pump 132 for collecting the sample gas from the gas flow via the suction tube 131 and the reservoir tank 133 for reserving the collected sample gas. The analyzer 14 may include the sensor 147 including a sensing device that outputs a detection signal corresponding to a concentration of a predetermined gas, and the third air pump 141 that supplies a predetermined volume of the sample gas from the reservoir tank 133 to the sensor 147.

According to the configuration described above, the sample gas collected by the gas collector 13 is reserved in the reservoir tank 133. Then, the predetermined volume of the sample gas is supplied from the reservoir tank 133 to the chamber 143 of the analyzer 14. By temporarily reserving the collected sample gas in the reservoir tank 133, the sample gas to be supplied to the analyzer 14 can be homogenized. According to this configuration, the volume of the sample gas to be supplied to the analyzer 14 is constant, which can further improve the analysis accuracy of the sample gas.

As described above, the analysis apparatus 1 may include the subject detector 11 and the controller 16 that controls the operation of the components of the analysis apparatus 1 and acquires detection signals from the sensor 147. The controller 16 may activate the first air pump 125 after a predetermined time has elapsed since the subject detector 11 detected the subject, and then activate the second air pump 132.

According to the above configuration, the analysis apparatus 1 generates a gas flow in the toilet bowl 4A after a predetermined time has elapsed since the subject seated on the toilet bowl 4A is detected, and then collects the sample gas. Thus, the analysis apparatus 1 can efficiently collect the sample gas.

The controller 16 may acquire the detection signal each time a predetermined volume of the sample gas and a predetermined volume of the purge gas are alternately supplied to the sensor 147. According to this configuration, the analysis apparatus 1 can repeatedly analyze the components of the sample gas multiple times, thereby improving the accuracy of the analysis result of the sample gas.

The analysis apparatus 1 may further include the fourth air pump 142 that supplies a purge gas for removing the sample gas after the detection processing by the sensor 147. According to this configuration, the sample gas after the detection processing by the sensor 147 can be removed using the purge gas. Thus, the analysis apparatus 1 can analyze a sample gas newly supplied to the sensor 147 without being affected by the sample gas after the detection processing.

The analysis apparatus 1 may include the blocker 15 near the gas collector 13 that blocks at least some of the gas flow. According to this configuration, a gas flow of a sample gas in the toilet bowl 4A is blocked near the gas collector 13, reducing a possibility that the sample gas flows out of the toilet bowl 4A. Thus, the sample gas can be efficiently collected from the gas flow.

When the toilet seat 4B corresponding to the toilet bowl 4A is located on the upper edge 4A1 of the toilet bowl 4A, the blocker 15 may be configured to close or narrow the gap P between the toilet seat 4B and the upper edge 4A1 of the toilet bowl 4A.

According to this configuration, the analysis apparatus 1 can efficiently collect the sample gas from the gas flow while avoiding the sample gas being discharged to the outside of the toilet bowl 4A.

Variations

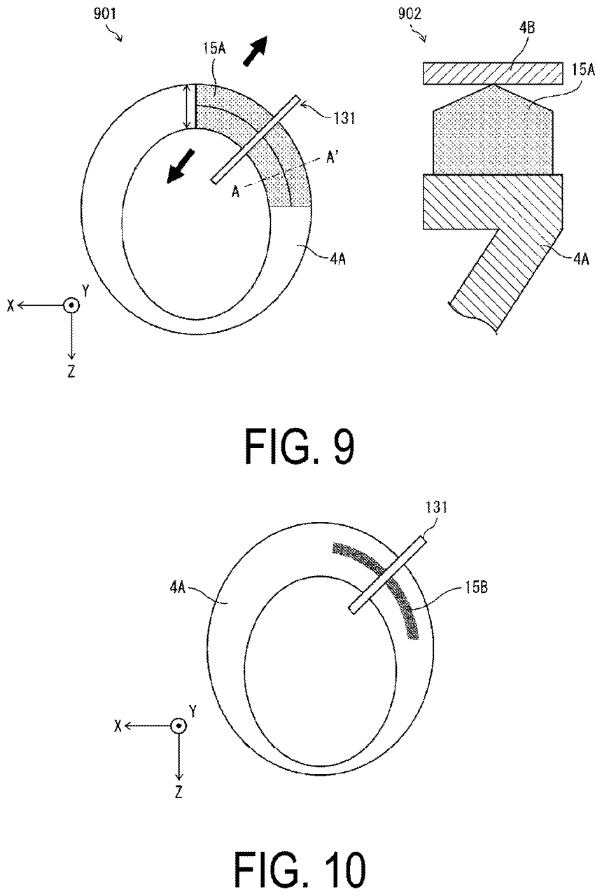

FIG. 9 is an external view illustrating a configuration of a blocker 15A, which is another example of the blocker 15. FIG. 10 is an external view illustrating a configuration of a blocker 15B, which is still another example of the blocker 15. Reference numeral 901 in FIG. 9 is a top view of the toilet bowl 4A, and reference numeral 901 is a cross-sectional view taken along line A-A′ in a diagram indicated in reference numeral 901. As illustrated in FIG. 9, the blocker 15A may be thinnest at an outer periphery and an inner periphery of the toilet bowl 4A, and the thickness increases toward the center between the outer periphery and the inner periphery of the toilet bowl 4A. As an example, as illustrated in reference numeral 901 in FIG. 9, the blocker 15A may have a pentagonal cross section. As illustrated in FIG. 10, the blocker 15B may be a configuration provided only partially in a width direction of the toilet bowl 4A.

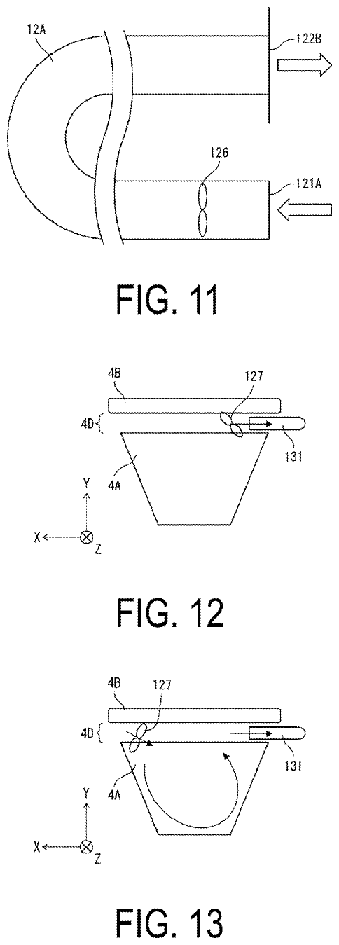

FIG. 11 is a schematic diagram illustrating another example of the gas flow generator 12. As illustrated in FIG. 11, a gas flow generator 12A, which is another example of the gas flow generator 12, may include a first blower fan 126 instead of the first air pump 125 as another example of the gas flow generating device 124. As an example, the first blower fan 126 may be a common fan that blows air by sucking gas from one surface and discharging air from an opposite surface of the one surface.

In the embodiment described above, the opening portion of the suction tube 131 is located near the gas flow generator 12. However, the position of the opening portion of the suction tube 131 is not limited thereto. For example, the opening portion of the suction tube 131 may be located on a wall surface of the pipe 123 of the gas flow generator 12. In this case, the opening portion of the suction tube 131 is provided with a valve that is normally closed. When a gas flow is generated by the gas flow generator 12, a sample gas can be sucked from the suction tube 131 by opening this valve.

In the embodiment described above, the gas collector 13 and the analyzer 14 include the second air pump 132, the third air pump 141, and the fourth air pump 142 for sucking, storing, or discharging a sample gas and a purge gas. However, the configuration of the gas collector 13 and the analyzer 14 is not limited thereto. For example, instead of the second air pump 132, the gas collector 13 may include a third blower fan capable of blowing gas in any direction. Instead of the third air pump 141 and the fourth air pump 142, the analyzer 14 may include two or more blower fans capable of blowing gas in any direction (e.g., a fourth blower fan and a fifth blower fan), respectively.

In the embodiment described above, the gas flow generator 12 may include a heater capable of warming gas passing through the pipe 123. Thus, air discharged by the gas flow generator 12 becomes warm air. Therefore, the possibility of discomfort caused by airflow generated by the airflow generator 12 touching the examinee's body can be reduced.

In the embodiment described above, the controller 16 activates the first air pump 125 to generate a gas flow after a predetermined time has elapsed since the examinee defecated. However, the timing to start generating the gas flow is not limited thereto. For example, the controller 16 may start generating the gas flow before the examinee defecates. To be more specific, in the flow of processing illustrated in FIG. 8, after S1, the processing of activating the first air pump 125 of S4 may be executed and then the processing of S2 may be executed. According to this processing, a gas flow is generated in the toilet 4 before the examinee defecates, so a sample gas is immediately swirled up when the examinee defecates. Therefore, the timing of suction of the sample gas by the gas collector 13 can be advanced.

The toilet 4 on which the analysis apparatus 1 is placed may be provided with a deodorizing device having a deodorizing function. In this case, the deodorizing device may activate the deodorizing function after the sample gas is completely sucked by the gas collector 13 in the analysis apparatus 1.

### Second Embodiment

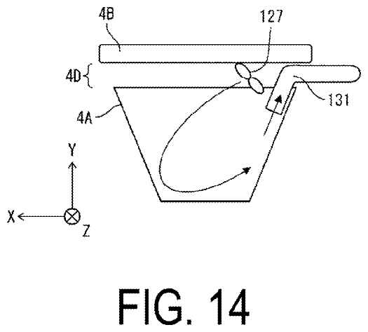

Another embodiment of the present disclosure will be described below. For the sake of convenience of description, members having the same functions as those of the members described in the above-described embodiment are denoted by the same reference signs, and description thereof is not repeated. FIG. 12 is a cross-sectional view of a toilet 4 including a gas flow generator 12A according to another embodiment. FIG. 12 is a cross-sectional view of the toilet 4 cut so as to include a suction tube 131. FIG. 13 is a cross-sectional view illustrating another example of the gas flow generator 12A. FIG. 14 is a cross-sectional view illustrating still another example of the gas flow generator 12A.

As illustrated in FIG. 12, an analysis apparatus 1A may include a second blower fan 127 as the gas flow generator 12A. The second blower fan 127 is a fan that generates a gas flow by sucking air from one surface and discharging air from an opposite surface, and blows air in a toilet bowl 4A. The second blower fan 127 includes a first surface 121B serving as a sucker 121 on the suction side and a second surface 122B serving as a discharger 122 on an opposite side of the first surface 121B.

The second blower fan 127 may be provided at any position in the toilet bowl 4A. As an example, as illustrated in FIG. 12, the second blower fan 127 may be provided near a suction tube 131 such that the second surface 122B faces the suction tube 131. As illustrated in FIG. 12, the second blower fan 127 may be provided slightly inclined from a direction parallel to an X-axis direction toward a −Y-axis direction. According to this configuration, the second blower fan 127 can be operated to generate a gas flow such that gas at a bottom of the toilet bowl 4A is directed toward the suction tube 131. According to this configuration, a sample gas in the toilet bowl 4A can be used to generate a gas flow in the toilet bowl 4A.

As another example, as illustrated in FIG. 13, the second blower fan 127 may be located on an opposite side of the suction tube 131 in the cross section of the toilet bowl 4A, and may be provided such that the second surface 122B faces an inner surface of the toilet bowl 4A. The second blower fan 127 may be provided slightly inclined from the direction parallel to the X-axis direction toward the −Y-axis direction. According to this configuration, by operating the second blower fan 127, the gas at the bottom of the toilet bowl 4A is swirled up to generate a gas flow toward the suction tube 131.

As still another example, as illustrated in FIG. 14, the second blower fan 127 may be located near the suction tube 131 in the cross section of the toilet bowl 4A, and may be provided such that the second surface 122B faces the inner surface of the toilet bowl 4A. The second blower fan 127 may be provided slightly inclined from the direction parallel to the X-axis direction toward the −Y-axis direction. The suction tube 131 may be placed slightly inclined from the direction parallel to the X-axis direction toward the −Y-axis direction. According to this configuration, by operating the second blower fan 127, the gas at the bottom of the toilet bowl 4A is swirled up to generate a gas flow toward the suction tube 131.

As described above, the second blower fan 127 provided as the gas flow generator 12A can be used to suck gas in the toilet bowl 4A, and discharges the gas, thereby generating a gas flow that circulates gas. Therefore, a possibility that air outside the toilet bowl 4A flows into the toilet bowl 4A and a concentration of the sample gas decreases is reduced. The concentration of the sample gas around the suction tube 131 can be increased by providing the second blower fan 127 as described above, so that a concentration of the gas contained in the sample gas can be measured with higher accuracy.

### Third Embodiment

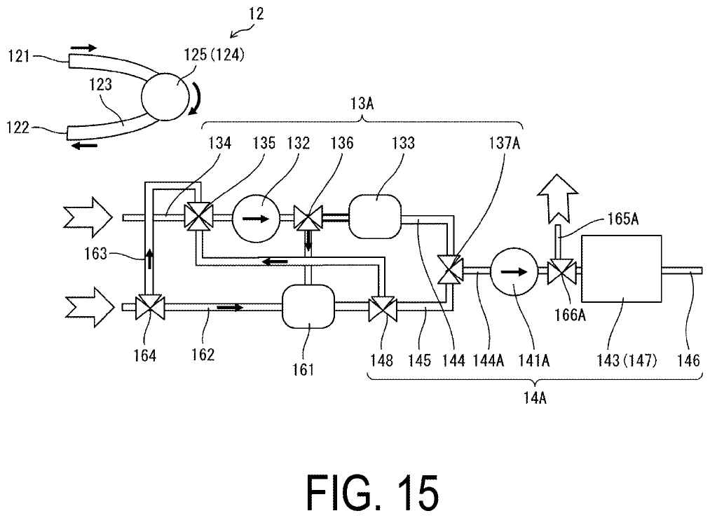

FIG. 15 is a schematic diagram illustrating a configuration of a gas collector 13A and an analyzer 14A according to another embodiment. As illustrated in FIG. 15, in the gas collector 13A and the analyzer 14A, the third air pump 141 and the fourth air pump 142 may be implemented by one air pump. To be specific, as illustrated in FIG. 15, the analyzer 14A may include a third air pump 141A instead of the third air pump 141 and the fourth air pump 142. The third air pump 141A is an air pump that serves as both the third air pump 141 and the fourth air pump 142, and is connected to a reservoir tank 133, a chamber 143, and a flow path 145 for supplying a purge gas. As illustrated in FIG. 15, in the configuration including the third air pump 141A, a flow path 144 and the flow path 145 merge upstream of the third air pump 141A, and a flow path 144A is provided between a junction of the flow path 144 and the flow path 145 and the third air pump 141A. At a junction of the flow path 144, the flow path 145, and the flow path 144A, a valve 137A capable of switching a connection state between the flow paths is provided.

The controller 16 can control the state of the valve 137A to allow the flow path 144 and the flow path 144A to communicate with each other. In this state, the controller 16 can supply a sample gas from the reservoir tank 133 to the chamber 143 by driving the third air pump 141A. The controller 16 can control the state of the valve 137A to allow the flow path 145 and the flow path 144A to communicate with each other. In this state, the controller 16 can supply a purge gas from a purge gas reservoir tank 161 to the chamber 143 by driving the third air pump 141A.

### Fourth Embodiment

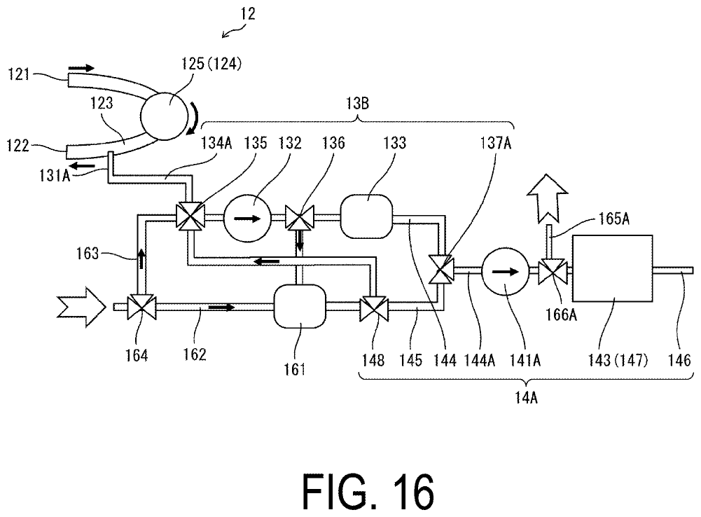

FIG. 16 is a schematic diagram illustrating a configuration of a gas flow generator 12, a gas collector 13B, and an analyzer 14A included in an analysis apparatus 1 according to another embodiment. As illustrated in FIG. 16, the gas collector 13B has a configuration in which an opening portion of a suction tube 131A is connected to a pipe 123 of the gas flow generator 12. According to this configuration, by driving a second air pump 132 while a first air pump 125 of the gas flow generator 12 is being driven to generate a gas flow, a sample gas is sucked from the pipe 123 to the suction tube 131A. The sucked sample gas further passes through a flow path 134A and is reserved in a reservoir tank 133.

### Fifth Embodiment

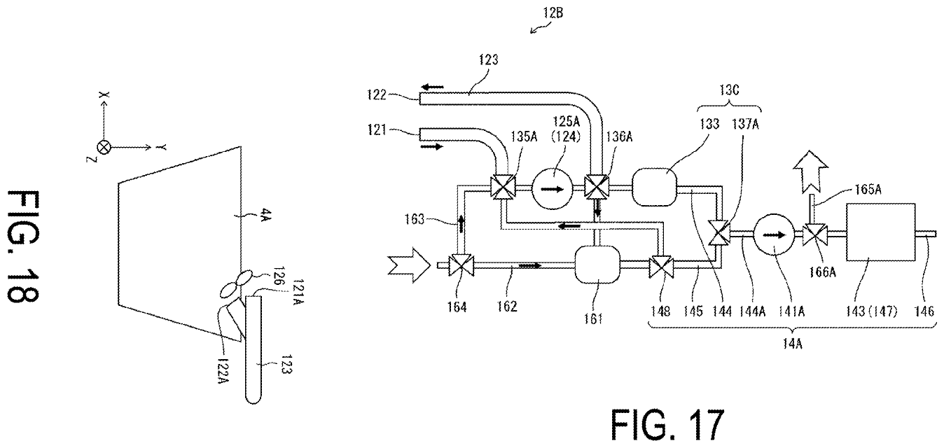

FIG. 17 is a schematic diagram illustrating a configuration of a gas flow generator 12B, a gas collector 13C, and an analyzer 14A according to another embodiment. As illustrated in FIG. 17, in the gas flow generator 12B and the gas collector 13C, the first air pump 125 as the gas flow generating device 124 and the second air pump 132 may be implemented by one air pump. To be more specific, as illustrated in FIG. 17, the gas collector 13C includes a first air pump 125A, which is an air pump that functions as the first air pump 125 and the second air pump 132.

A sucker 121 of the gas flow generator 12B is connected to a valve 135A between the first air pump 125A and a flow path 163, and the controller 16 can change a connection state of the flow paths by switching an open state of the valve 135A. A discharger 122 of the gas flow generator 12B is connected to a valve 136A between the first air pump 125A and a reservoir tank 133, and the controller 16 can change a connection state of the flow paths by switching an open state of the valve 136A. Other configurations of the gas flow generator 12B, the gas collector 13C, and the analyzer 14A are the same as those of the gas flow generator 12, the gas collector 13A, and the analyzer 14A illustrated in FIG. 15.

In the configuration described above, by driving the first air pump 125A with the valve 135A and the valve 136A open and other valves closed, air in a toilet 4 is sucked from the sucker 121 and discharged from the discharger 122. Thus, a gas flow can be generated in the toilet 4. By further adjusting the open states of the valves while the gas flow is being generated in the toilet 4, a sample gas flowing in the gas flow generator 12B can be supplied to the reservoir tank 133 and reserved therein.

By opening the valve 135A, the valve 136A, and a valve 164 and driving the first air pump 125A, a purge gas is sucked from the outside and reserved in a purge gas reservoir tank 161 through the flow path 163.

Example of Software Implementation

Functions of the analysis apparatus 1 (hereinafter referred to as “apparatus”) can be implemented by a program for causing a computer to function as the apparatus and for causing the computer to function as the controller 16 of the apparatus.

In this case, the apparatus includes a computer including at least one control device (e.g., processor) and at least one storage device (e.g., memory) as hardware for executing the program. By executing the program by the control device and the storage device, the functions described in the embodiments are implemented.

The program may be recorded on one or more computer-readable non-transitory recording media. The recording media may or may not be included in the apparatus. In the latter case, the program may be supplied to the apparatus via any wired or wireless transmission medium.

Some or all of the functions of the control blocks can be implemented by logic circuits. For example, an integrated circuit in which logic circuits functioning as the control blocks are formed is also included in the scope of the present disclosure. In addition to this, for example, a quantum computer can implement the functions of the control blocks.

The several types of processing described in the embodiments may be executed by artificial intelligence (AI). In this case, the AI may operate in the control device, or may operate in another device (e.g., an edge computer or a cloud server).

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, a person skilled in the art can easily make various variations or modifications based on the present disclosure. Note that these variations or modifications are included within the scope of the present disclosure.

FIG. 18 is a schematic diagram illustrating another example of the gas flow generator 12. As illustrated in FIG. 18, the analysis apparatus 1 may include, as the gas flow generator 12, both the pipe 123 connecting the first end 121A and the second end 122A and the second blower fan 127. As illustrated in FIG. 18, the second blower fan 127 may be placed near the first end 121A so as to cause gas to flow from the toilet bowl 4A to the first end 121A. The second blower fan 127 may be placed at a position away from the first end 121A. For example, the second blower fan 127 may be placed at a position as illustrated in FIG. 13 or 14. In this case, the first end 121A may be placed near the suction tube 131.

### EXAMPLES

Examples of the disclosure will be described below. In the following examples, a structure simulating the toilet bowl 4A was prepared, and the analysis apparatus 1 was mounted to this structure to measure a gas concentration. Hereinafter, this structure is also referred to as “toilet bowl 4A”.

### Example 1

In this example, CO₂ gas of 6000 ppm was blown out from the bottom of the toilet bowl 4A at 200 ml/min, and the CO₂ concentration was measured using the analysis apparatus 1. The suction tube 131 was placed on the upper edge 4A1 of the toilet bowl 4A.

As a comparative example, as indicated in reference numeral 701 in FIG. 7, a sample gas was sucked from the suction tube 131 without providing the blocker 15, and a CO₂ concentration was measured. As an example, as indicated in reference numeral 702 in FIG. 7, a sample gas was sucked from the suction tube 131 with the blocker 15 provided around the suction tube 131, and the CO₂ concentration was measured. The CO₂ concentration was measured from 0 seconds to 900 seconds, with a start of CO₂ supply to the toilet bowl 4A set to 0 seconds.

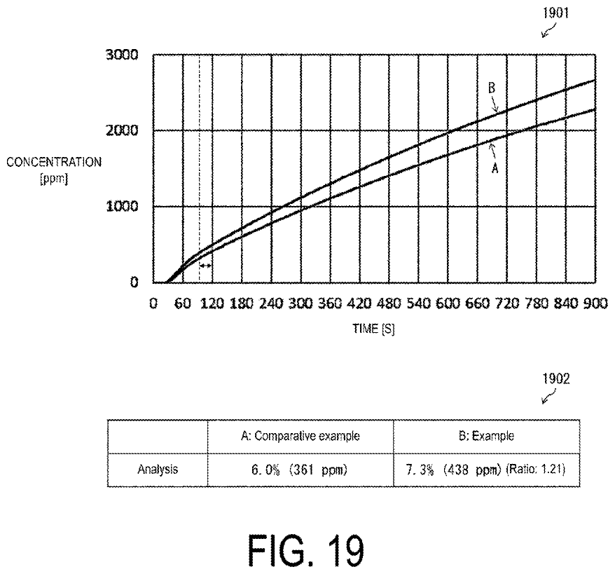

FIG. 19 includes a graph and a table showing results of experiments according to Example 1. Reference numeral 1901 in FIG. 19 is a graph showing the results of the measurements described above. The horizontal axis represents time when the start of CO₂ supply to the toilet bowl 4A is set to 0, and the vertical axis represents the CO₂ concentration (ppm) detected by the sensor 147.

In the experiments described above, the mean value of the concentration values from 90 seconds to 120 seconds in each example was calculated, and the ratio to the CO₂ concentration actually supplied to the toilet bowl 4A was calculated.

Reference numeral 1902 in FIG. 19 is a table showing this ratio. This ratio indicates a relative suction efficiency of the sample gas sucked from the suction tube 131 toward the sensor 147. As shown in FIG. 19, when the blocker 15 was provided around the suction tube 131, the suction efficiency was improved compared to when the blocker 15 was not provided. As described above, the analysis apparatus 1 was able to increase the absorption efficiency of the sample gas by providing the blocker 15 around the suction tube 131.

### Example 2

In this example, CO₂ gas of 6000 ppm was blown out from the bottom of the toilet bowl 4A at 200 ml/min, and a CO₂ concentration was measured using the analysis apparatus 1. The suction tube 131 was placed on the upper edge 4A1 of the toilet bowl 4A.

As Example A, a sample gas was sucked from the suction tube 131 without providing the gas flow generator 12, and the CO₂ concentration was measured. As each of Examples B to E, the second blower fan 127 as the gas flow generator 12 was provided in the toilet bowl 4A in a configuration as illustrated in FIG. 12, a sample gas was sucked after the second blower fan 127 was driven, and the CO₂ concentration was measured. The measurement was performed from 0 seconds to 180 seconds, with the start of driving of the second blower fan 127 set to 0 seconds. The measurements were performed after driving the second blower fan 127 at a drive voltage of 1.5 V in Example B, 2.2 V in Example C, 3.0 V (rated voltage) in Example D, and 4.5 V in Example E.

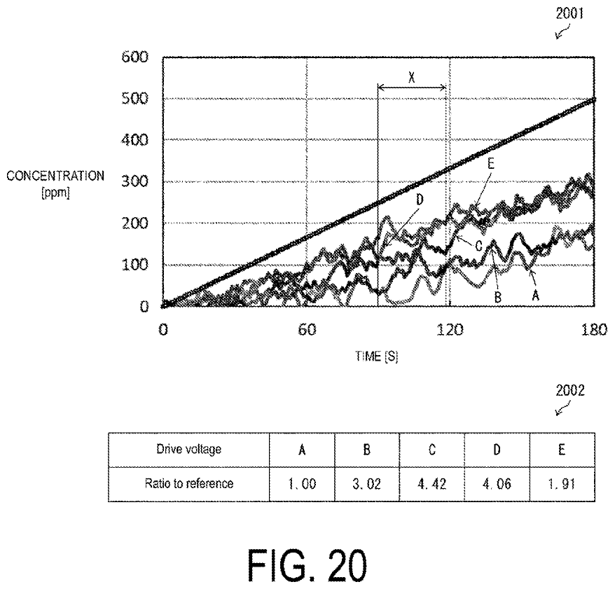

FIG. 20 includes a graph and a table showing results of experiments according to Example 2. Reference numeral 2001 in FIG. 20 is a graph showing the results of the measurements described above. A horizontal axis represents time when the start of driving of the second blower fan 127 is set to 0, and a vertical axis represents the CO₂ concentration (ppm) detected by the sensor 147.

In the experiments described above, the mean value of the measured values from 90 seconds to 120 seconds for each example was calculated, and the ratio of each example to Example A was calculated.

Reference numeral 2002 in FIG. 20 is a table showing this ratio. This ratio reflects the accuracy of sample gas detection in each example compared to Example A. In other words, the ratio indicates the relative suction efficiency of the sample gas sucked from the suction tube 131 toward the sensor 147. As shown in this table, when the second blower fan 127 was provided as the gas flow generator 12, the suction efficiency was improved compared to when the second blower fan 127 was not provided. In particular, in Examples B to D, that is, examples in which the second blower fan 127 was driven at a voltage from 1.5 V to 3.0 V, the absorption efficiency was increased three times or more. As described above, the analysis apparatus 1 was able to improve the absorption efficiency of the sample gas by including the gas flow generator 12.

### Example 3

In this example, CO₂ gas of 6000 ppm was blown out from the bottom of the toilet bowl 4A at 200 ml/min, and the CO₂ concentration was measured using the analysis apparatus 1. The suction tube 131 was placed on the upper edge 4A1 of the toilet bowl 4A.

As Example A, a sample gas was sucked from the suction tube 131 without providing the gas flow generator 12, and the CO₂ concentration was measured. As each of Examples B to E, the second blower fan 127 as the gas flow generator 12 was provided in the toilet bowl 4A in a configuration as illustrated in FIG. 12, a sample gas was sucked after the second blower fan 127 was driven, and the CO₂ concentration was measured. The measurement was performed from 0 seconds to 180 seconds, with the start of driving of the second blower fan 127 set to 0 seconds. In this example, when the toilet bowl 4A was viewed from the top, a position where the suction tube 131 was provided and a gas discharge port for discharging a sample gas were connected by a straight line, and the second blower fan 127 was placed at a position where the straight line was in contact with the toilet 4. Measurement was performed using this positional relationship as a reference (Example A). The suction tube 131 was moved along an edge of the toilet bowl 4A to a back side (+Z-axis direction in the drawing indicated in reference numeral 201 in FIG. 2) and to a front side (−Z-axis direction in the drawing indicated in reference numeral 201 in FIG. 2) to perform further measurement as Example B to Example D. The second blower fan 127 in Example B was placed by moving 1 cm from the reference to the back. The second blower fan 127 in Example C was placed at the reference position. The fan in Example D was placed by moving 1 cm from the reference to the front. In Example B, Example C, and Example D, the measurements were performed while the second blower fan 127 was driven at a drive voltage of 2.2 V. In Example A, the measurement was performed without driving the second blower fan 127.

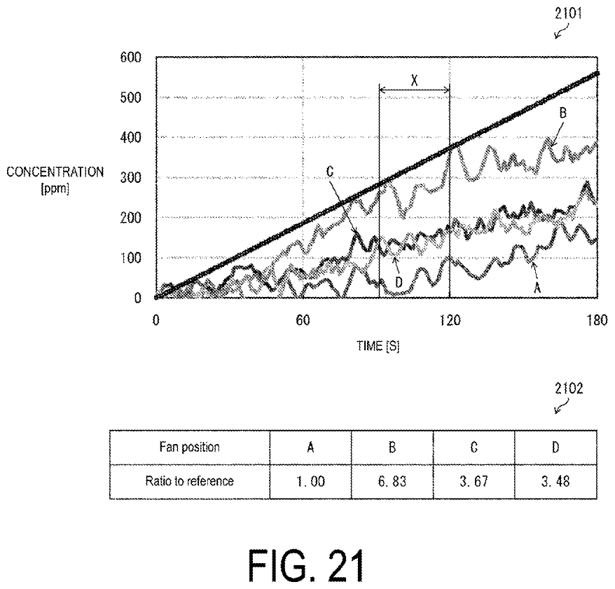

FIG. 21 is a graph and a table showing results of experiments according to Example 3. Reference numeral 2101 in FIG. 21 is a graph showing the results of the measurements described above. A horizontal axis represents the time when the start of driving the second blower fan 127 is set to 0, and a vertical axis represents the CO₂ concentration (ppm) detected by the sensor 147.

In the experiments described above, the mean value of the measured values from 90 seconds to 120 seconds for each example was calculated, and the ratio of each example to Example A was calculated.

Reference numeral 2102 in FIG. 21 is a table showing this ratio. This ratio reflects the accuracy of sample gas detection in each example compared to Example A. In other words, the ratio indicates the relative suction efficiency of the sample gas sucked from the suction tube 131 toward the sensor 147. As shown in this table, when the second blower fan 127 was driven as the gas flow generator 12, the suction efficiency was improved compared to when the second blower fan 127 was not driven. In particular, in Example B, the absorption efficiency was improved by more than six times. As described above, the analysis apparatus 1 was able to improve the absorption efficiency of the sample gas by including the gas flow generator 12. By setting the positional relationship between the suction tube 131 and the second blower fan 127 as in Example B, the absorption efficiency of the sample gas was further improved.

### Example 4

In this example, a specimen (feces) that emits a sample gas was placed at the bottom of the toilet bowl 4A, and a CO₂ mass fraction in the toilet bowl 4A was analyzed for each predetermined time. As an example, the gas flow generator 12 including the first end 121A, the second end 122A, and the pipe 123 was provided on the toilet bowl 4A in the configuration shown in FIG. 5 and operated, then, the CO₂ mass fraction was analyzed for each predetermined time. To be specific, the first end 121A was located near the suction tube 131, and the second end 122A was located at a position away from the suction tube 131. The gas flow generator 12 was operated at a discharge and suction rate of 2 L/min. As a comparative example, the CO₂ mass fraction was analyzed for each predetermined time in the toilet bowl 4A under the same conditions except that the gas flow generator 12 was not provided.

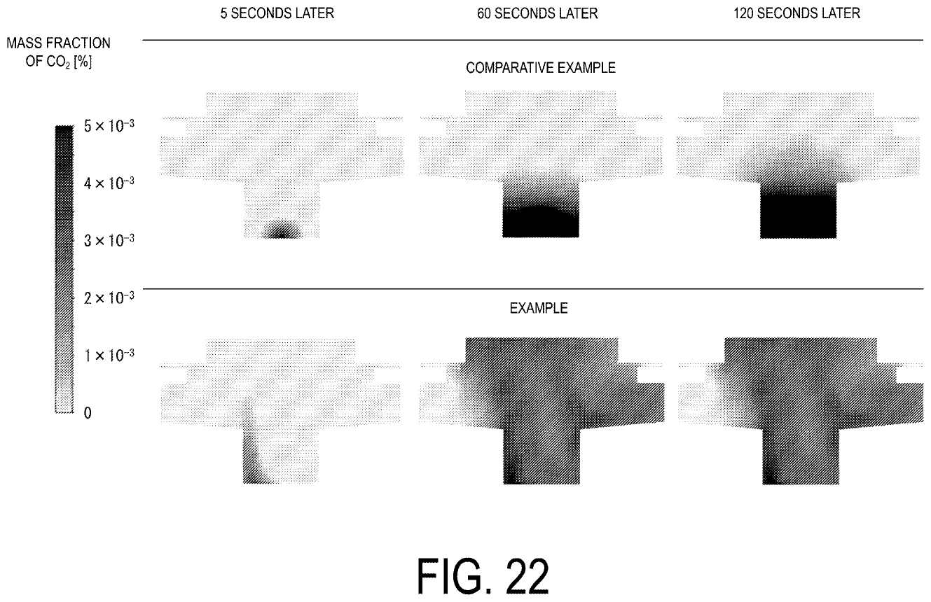

FIG. 22 includes diagrams, each illustrating the mass fraction distribution of CO₂ in the toilet bowl 4A, which is a result of the analysis according to Example 4. As illustrated in FIG. 22, in the comparative example, the CO₂ mass fraction at an upper portion of the toilet bowl 4A remained low even after the time elapsed since the specimen was placed. On the other hand, in the example, CO₂ at the bottom of the toilet bowl 4A was swirled up due to the gas flow generated by the gas flow generator 12, and the CO₂ mass fraction at the upper portion of the toilet bowl 4A increased. In particular, as illustrated in FIG. 22, in the example, the CO₂ mass fraction increased around a position where the suction tube 131 was provided. As described above, the analysis apparatus 1 was able to increase the concentration of the sample gas at a desired position by including the gas flow generator 12 including the first end 121A, the second end 122A, and the pipe 123.

### Example 5

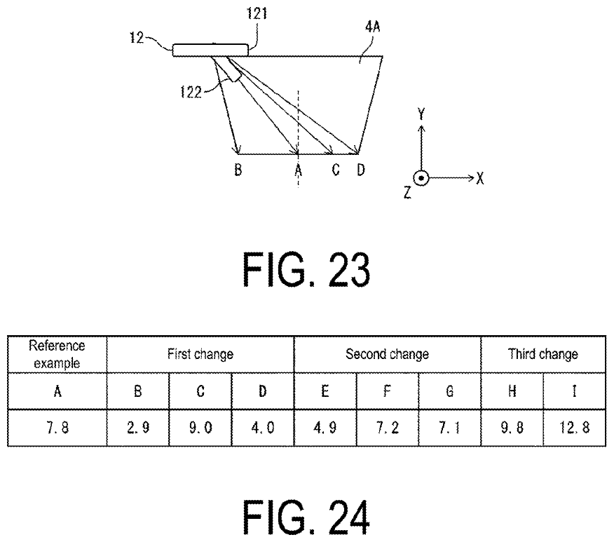

FIG. 23 is a schematic diagram illustrating an example of arrangement of the gas flow generator 12 in experiments according to Example 5. In this example, a specimen (feces) that emits CO₂ as a sample gas was placed at the bottom of the toilet bowl 4A to which the analysis apparatus 1 was mounted, and the yield of CO₂ by the analysis apparatus 1 was calculated by comparing the total amount of CO₂ emitted from the specimen and the total amount of CO₂ absorbed by the analysis apparatus 1. The following formula (1) was used to calculate the yield.

Yield=(total amount of CO₂ sucked into the reservoir tank)/(total amount of CO₂ emitted from the specimen) (1)

First, as a reference example, as illustrated in FIG. 23, the analysis apparatus 1 including the gas flow generator 12 including the sucker 121 and the discharger 122 was mounted on the toilet bowl 4A, and the gas flow generator 12 was driven at a gas flow rate of 2 L/min to suck a sample gas. The yield in Example A was calculated with the reference example as Example A. In Example A, as illustrated in FIG. 23, the gas flow generator 12 was placed so that the axis B of the discharger 122 passes through the center of the bottom of the toilet bowl 4A in the X-axis direction (the position indicated by symbol A in FIG. 23) when the toilet bowl 4A was viewed from the rear. In Example A, when the toilet bowl 4A was viewed from the top, the gas flow generator 12 was placed such that a line connecting the center point of the bottom of the toilet bowl 4A in the X-axis direction and the Z-axis direction and the center point between the openings as the sucker 121 and the discharger 122 of the gas flow generator 12 is parallel to the X-axis. In Example A, the suction tube 131 was placed near the sucker 121.

Subsequently, as a first change, a sample gas was sucked using the analysis apparatus 1 in which the angle of the axis B of the discharger 122 was changed from the configuration in Example A, and the yield was calculated. In Example B, the gas flow generator 12 was placed such that the axis B passed through an end portion of the bottom of the toilet bowl 4A in the −X-axis direction (a position indicated by symbol B in FIG. 23). In Example C, the gas flow generator 12 was placed such that the axis B passed through a position at ¾ in the +X-axis direction from the end portion of the bottom of the toilet bowl 4A in the −X-axis direction (a position indicated by symbol C in FIG. 23). In Example D, the gas flow generator 12 was placed such that the axis B passed through an end portion of the bottom of the toilet bowl 4A in the +X-axis direction (a position indicated by symbol D in FIG. 23).

As a second change, a sample gas was sucked using the analysis apparatus 1 in which the position of the gas flow generator 12 was changed from the configuration in Example A, and the yield was calculated. In Example E, the gas flow generator 12 was moved 2 cm in the −Z-axis direction when the toilet bowl 4A was viewed from the top. In Example F, the gas flow generator 12 was moved 4 cm in the −Z-axis direction when the toilet bowl 4A was viewed from the top. In Example G, the gas flow generator 12 was moved 2 cm in the +Z-axis direction when the toilet bowl 4A was viewed from the top.

As a third change, a sample gas was sucked using the analysis apparatus 1 in which the position of the suction tube 131 of the analysis apparatus 1 was changed, and the yield was calculated. In Example H, the suction tube 131 was moved 2.5 cm in the −Z-axis direction from the position in Example A so that the suction tube 131 was placed between the sucker 121 and the discharger 122. In Example I, the suction tube 131 was moved 2.5 cm in the −Z-axis direction from the position in Example A so that the suction tube 131 was placed between the sucker 121 and the discharger 122, and further moved another 1 cm in the X-axis direction.

FIG. 24 is a table showing results of the experiments according to Example 5. As shown in FIG. 24, by configuring the discharger 122 as in Example C, the yield of the sample gas was improved. The arrangement of the gas flow generator 12 as in Example A, Example F, and Example G improved the yield of the sample gas more than the arrangement of the gas flow generator 12 as in Example E. By locating the suction tube 131 as in Example I, the yield of the sample gas was improved. As described above, the yield of the sample gas was able to be improved by appropriately adjusting the arrangement of components of the analysis apparatus 1.

### REFERENCE SIGNS

1, 1A Analysis apparatus
2 Server apparatus
3 Electronic device
4 Toilet
4A Toilet bowl
4A1 Upper edge
4B Toilet seat
11 Subject detector
12, 12A Gas flow generator
13, 13A, 13B, 13C Gas collector (Sampler)
14, 14A, 14B Analyzer
15, 15A Blocker
16 Controller
100 Analysis system
121 Sucker
122 Discharger
123 Pipe section
124 Gas flow generating device
125, 125A First air pump
126 First blower fan
127 Second blower fan
132 Second air pump
133 Reservoir tank (Reservoir)
141, 141A Third air pump
142 Fourth air pump
147 Sensor
P Gap

1. An analysis apparatus comprising:
a gas flow generator comprising a sucker configured to suck a sample gas in a toilet bowl and a discharger configured to discharge the sample gas sucked by the sucker toward the inside of the toilet bowl, and configured to generate a gas flow of the sample gas passing through the inside of the toilet bowl; and
an analyzer configured to analyze a component contained in the sample gas.

2. The analysis apparatus according to claim 1, wherein
the gas flow generator comprises
a pipe comprising a first end being the sucker and a second end being the discharger, and
a gas flow generating device located in the pipe, and
the first end and the second end are directed into the toilet bowl.

3. The analysis apparatus according to claim 2, wherein
the gas flow generating device is a first air pump or a first blower fan.

4. The analysis apparatus according to claim 1, wherein
the gas flow generator comprises a second blower fan configured to blow air in the toilet bowl near an upper edge of the toilet bowl.

5. The analysis apparatus according to claim 1, further comprising:
a sampler configured to collect at least some of the gas flow as the sample gas.

6. The analysis apparatus according to claim 5, wherein
the sampler further comprises a reservoir configured to reserve the sample gas collected.

7. The analysis apparatus according to claim 5, wherein
the sampler comprises a second air pump or a third blower fan configured to collect the sample gas from the gas flow.

8. The analysis apparatus according to claim 1, comprising:
a reservoir configured to reserve the sample gas, wherein
the analyzer comprises
a sensor comprising a sensing device configured to output a detection signal corresponding to a concentration of a predetermined gas, and
a third air pump or a fourth blower fan configured to supply a predetermined volume of the sample gas from the reservoir to the sensor.

9. The analysis apparatus according to claim 1, wherein
the gas flow generator comprises
a pipe comprising a first end being the sucker and a second end being the discharger,
a gas flow generating device located in the pipe,
a sampler configured to collect at least some of the gas flow as the sample gas, and
a reservoir configured to reserve the sample gas collected,
the first end and the second end are directed into the toilet bowl,
the sampler comprises a second air pump or a third blower fan configured to collect the sample gas from the gas flow via the sampler, and
the analyzer comprises
a sensor comprising a sensing device configured to output a detection signal corresponding to a concentration of a predetermined gas, and
a third air pump or a fourth blower fan configured to supply a predetermined volume of the sample gas from the reservoir to the sensor.

10. The analysis apparatus according to claim 1, comprising:
a sampler configured to collect at least some of the gas flow as the sample gas, wherein
the gas flow generator comprises a second blower fan comprising the sucker and the discharger,
the second blower fan is directed into the toilet bowl,
the sampler comprises
a second air pump or a third blower fan configured to collect the sample gas from the gas flow via the sampler, and
a reservoir configured to reserve the sample gas collected, and
the analyzer comprises
a sensor comprising a sensing device configured to output a detection signal corresponding to a concentration of a predetermined gas, and
a third air pump or a fourth blower fan configured to supply a predetermined volume of the sample gas from the reservoir to the sensor.

11. The analysis apparatus according to claim 9, comprising:
a subject detector configured to detect a subject seated on the toilet bowl; and
a controller configured to control (1) the gas flow generator, (2) the second air pump or the third blower fan, and (3) the third air pump or the fourth blower fan, and acquire the detection signal from the sensor, wherein
after a lapse of a predetermined time after a detection of the subject, the controller is configured to activate the gas flow generator, and then activate the second air pump or the third blower fan.

12. The analysis apparatus according to claim 8, comprising:
a subject detector configured to detect a subject seated on the toilet bowl; and
a controller configured to control (1) the gas flow generator and (2) the third air pump or the fourth blower fan, and acquire the detection signal from the sensor, wherein
after a lapse of a predetermined time after a detection of the subject, the controller is configured to activate the gas flow generator, and then reserve the sample gas in the reservoir.

13. The analysis apparatus according to claim 11, wherein
the controller is configured to acquire the detection signal each time the predetermined volume of the sample gas and the predetermined volume of a purge gas are alternately supplied to the sensor.

14. The analysis apparatus according to claim 13, further comprising:
a fourth air pump or a fifth blower fan configured to supply the purge gas to the sensor.

15. The analysis apparatus according to claim 1, comprising:
a sampler configured to collect at least some of the gas flow as the sample gas; and
a blocker configured to block at least another some of the gas flow near the sampler.

16. The analysis apparatus according to claim 15, wherein
when a toilet seat corresponding to the toilet bowl is located above an upper edge of the toilet bowl, the blocker is configured to close or narrow a gap between the toilet seat and the upper edge of the toilet bowl.

17. The analysis apparatus according to claim 1, comprising:
a sampler configured to collect at least some of the gas flow as the sample gas, wherein
when a toilet seat corresponding to the toilet bowl is placed above an upper edge of the toilet bowl, the sucker, the discharger, and the sampler are located between the toilet seat and the upper edge of the toilet bowl.

18. An analysis system comprising:
the analysis apparatus according to claim 11;
a server apparatus configured to estimate a health condition of the subject based on an analysis result obtained by the analysis apparatus and output health information corresponding to the health condition estimated; and
a terminal device configured to receive the health information from the server apparatus and present the health information to the subject.

19. Analysis method comprising:
generating a gas flow passing through the inside of a toilet bowl by sucking a sample gas in the toilet bowl and discharging the sample gas sucked toward the inside of the toilet bowl;
collecting the sample gas from the gas flow; and
analyzing a component contained in the sample gas collected.

20. A non-transitory computer-readable storage medium that stores a control program configured to control a computer, the control program executing processes causing the computer to:
function as the analysis apparatus according to claim 11, and
causing the computer to function as the controller.

21. (canceled)
